(19) United States
(12) Plant Patent Application Publication
(10) Pub. No.: US 2007/0261148 P1
(43) Pub. Date: Nov. 8, 2007

(54) OSTEOSPERMUM PLANT NAMED 'OSNEWI'

(75) Inventor: Anna M.W.P. Houbraken, Enkhuizen (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/430,148

(22) Filed: May 8, 2006

(51) Int. Cl. A01H 5/00

(52) U.S. Cl. PLT/360

(57) ABSTRACT

A new Osteospermum plant particularly distinguished by its compact round plant habit, early flowering with white colored ray florets, blue colored ray florets, very large and good quality inflorescences.

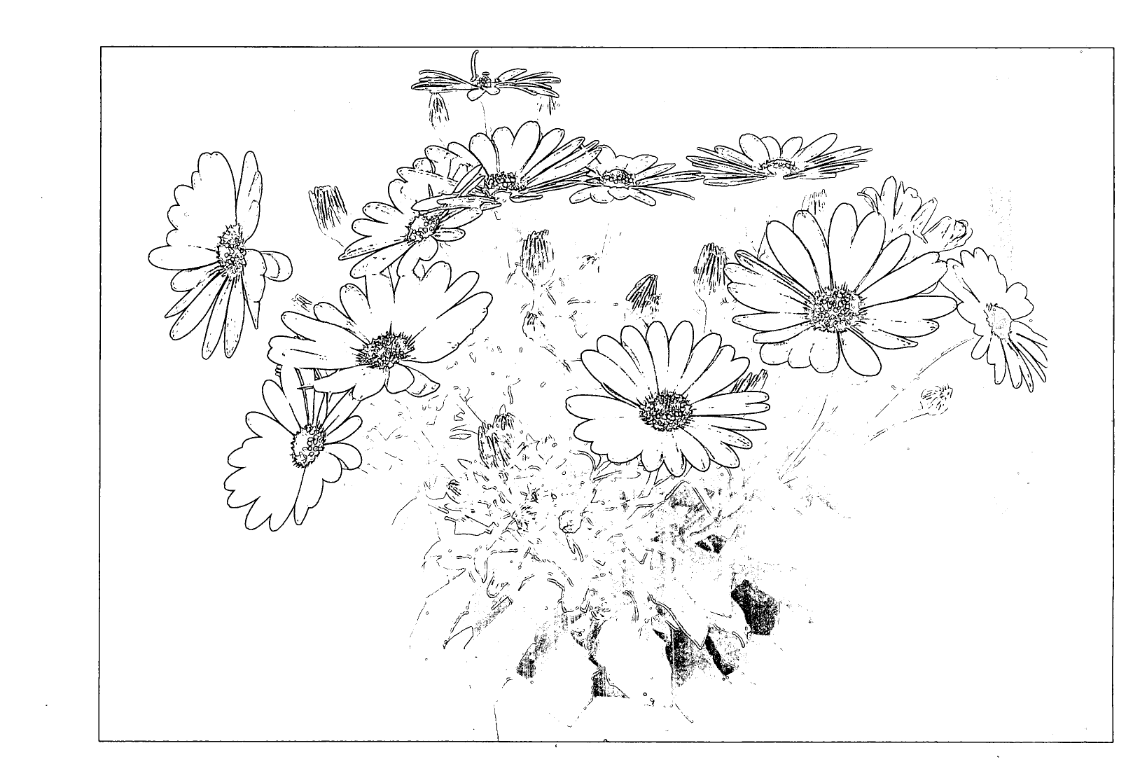

### LATIN NAME OF THE GENUS AND SPECIES OF THE PLANT CLAIMED

Osteospermum ecklonis

### VARIETAL DENOMINATION

‘Osnewi’

### BACKGROUND OF THE NEW PLANT

The present invention relates to a new and distinct cultivar of Osteospermum plant, botanically known as Osteospermum ecklonis.

The new Osteospermum is propagated by cuttings resulting from an open pollinated population from a proprietary Osteospermum ecklonis seedling selection as the female, or seed, parent identified as breeder's code ‘E0210-2’ (unpatented). Breeder's code of new plant is ‘F0195-2.’

The new Osteospermum was discovered and selected as a single flowering plant within the progeny of the stated population grown in Enkhuizen, Netherlands, in 2001. The new Osteospermum plant has been repeatably asexually reproduced by cuttings in Enkhuizen, Netherlands over a period of 4 years. It has been found to retain its distinctive characteristics through successive propagations, and this novelty appears to be firmly fixed.

### DESCRIPTION OF THE DRAWING

This new Osteospermum plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Osteospermum plant The data which defines these characteristics were collected from asexual reproductions carried out in Enkhuizen, Netherlands. The plant history was taken on 25 weeks old plants. The instant plant was grown indoors in a 10.5 cm container and transplanted into the open field in week 20.

Color references are primarily to the R.H.S. Colour Chart of The Royal Horticultural Society of London, edition 2001.

TABLE 1
DIFFERENCES BETWEEN THE NEW CULTIVAR
‘OSNEWI’ AND A SIMILAR CULTIVAR

| | | | ‘Osjamwhit’ (U.S. | |
|---|---|---|---|---|
| | ‘Osnewi’ | | Application No. 11/167,496) | |
| Plant height | 24 | cm | 20-22 | cm |
| Plant width | 26-30 | cm | 26 | cm |
| Flower quality | Overlapping petals | | No overlapping petals | |
| Flower size | 6.5 | cm | 5.5 | cm |

The plant:
   Classification.—Botanical: Osteospermum ecklonis.
Parentage:
   Female parent.—Proprietary seedling selection ‘E0210-2.’
   Male parent.—Female is open pollinated so male is unknown.
Plant description:
   Growth habit.—Round, compact.
   Plant height.—24 cm.
   Speading area of plant.—26-30 cm.
   Strength.—Moderate growth.
   Branching character.—Freely branching, 18 lateral branches without pinching.
   Blooming period.—From May until October.
The stem:
   Length.—16 cm.
   Diameter.—5 mm.
   Internode length.—5-10 mm.
   Texture.—Glabrous.
   Color.—RHS 144A.
The foliage:
   Shape.—Single leaves, alternate and elliptic, apex: broadly acute base: attenuate, margin: irregular serration.
   Number of leaves per lateral branch.—90.
   Texture.—Glabrous, leathery.
   Leaf.—Color — Upper side: RHS 137B Color — Under side: RHS 137C Length: Young 26-30 mm, mature 60-62 mm Width: Young 4-6 mm, mature 16-18 mm.
   Petiole.—Absent.
The inflorescence:
   Number of inflorescences per mature plant.—60.
   Inflorescence bud.—Form: Ovoid Length: 10-12 mm Width: 6-10 mm Color: RHS 151C.
   Inflorescence lastingness on the plant.—Around 5 days depending on the temperature.
   Inflorescence.—Diameter: 65 mm Disc diameter: 9 mm.
   Inflorescence depth.—8 mm.
   Fragrance.—No fragrance.
   Disc florets.—Shape: Tubular, elongated Apex: Five pointed Length: 8 mm Width: 1 mm Number of disc florets per inflorescence: 55 Color: RHS 99B.
   Ray florets.—Length: 28-30 mm Width: 9 mm Shape: Capitulum with one series of ray florets Color Upper surface: RHS 155A Middle surface: RHS ring RHS 70B Lower surface: RHS 186B Number of ray florets: 17-19 Shape — Apex: Rounded Base: Acute Margin: Entire Texture: Satiny.
   Peduncles.—Length: 5-8 cm Texture: Sturdy, tiny hairs Color: RHS 143C Diameter: 2 mm Shape: Round.
   Phyllaries.—Texture Rough, tiny hairs Number of phyllaries: 14-16 Color: RHS 143C Length: 10 mm Margin: Entire Width: 1 mm Apex: Acute Shape: Ligulate Color: RHS 137C both sides.
Reproductive organs:
   Androecium.—Appearance: Present on disc florets only Shape: Oblong, five stamens fuised to one Length: 2 mm Width: 1mm Pollen color: RHS 25A Total no. of anthers: 60.
   Gynoecium.—Appearance: Present on both ray and disc florets Pistil number: One pistil per ray or disc floret Pistil length: 4 mm Stigma color: RHS 77A Stigma shape: Bipartite.
Seed development: Seed development has been observed on plants of the new Osteospermum.
   Amount of seeds produced.—Moderate.
   Seed length.—5 mm.
   Diameter.—3 mm.
   Color.—RHS 200A.
   Shape.—Elliptic.
Disease resistance: Under commercial conditions, plants of the new Osteospermum are resistant to fungal pathogens common to Osteospermum.

What is claimed is:

1. A new and distinct variety of Osteospermum plant, substantially as illustrated and described herein.
